(19) United States
(12) Patent Application Publication
(10) Pub. No.: US 2026/0294502 A1
(43) Pub. Date: Oct. 1, 2026

(54) REFRIGERANT ELECTROMECHANICAL CONTROL

(71) Applicant: Cryotherapeutics SA, Awans (BE)

(72) Inventors: Stewart Maddison FOX, Cambridge (GB); Peter Georg LAITENBERGER, Cambridge (GB)

(73) Assignee: Cryotherapeutics SA, Awans (BE)

(21) Appl. No.: 19/489,379

(22) PCT Filed: Jun. 6, 2024
(86) PCT No.: PCT/EP2024/065667

§ 371 Date: Dec. 2, 2025

(30) Foreign Application Priority Data
Jun. 6, 2023 (GB) 2308402.3

(51) Int. Cl. A61B 18/02; A61B 18/00; A61B 90/00

(52) U.S. Cl. CPC A61B 18/02; A61B 90/06; A61B 2018/00642; A61B 2018/00666; A61B 2018/00714; A61B 2018/00726; A61B 2018/00732; A61B 2018/00744; A61B 2018/00791; A61B 2018/0212; A61B 2018/0262; A61B 2090/064

(57) ABSTRACT

The method includes supplying cryogenic fluid to a balloon of a balloon catheter in at least two phases, the at least two phases including a cooldown phase and a regulation phase subsequent to the cooldown phase. The cooldown phase includes coupling a source of the cryogenic fluid to the balloon; delivering the cryogenic fluid to the balloon via a flow control valve to cool the balloon; obtaining one or more temperature measurements for the balloon; determining that a temperature of the balloon satisfies a first condition; and, in response to the determining, closing the flow control valve. The regulation phase includes repeating obtaining one or more further temperature measurements for the balloon; determining whether a temperature of the balloon satisfies a second condition; and, if the temperature of the balloon satisfies the second condition, opening the flow control valve to increase supply of the cryogenic fluid to the balloon.

### FIELD

The present invention relates to methods of controlling temperature of a catheter, more specifically a balloon catheter.

### BACKGROUND TO THE INVENTION

Catheters for cryotherapy have been used in the cardiovascular system since the late 1970s. Over the ensuing years it became widely recognised that cryotherapy was particularly advantageous for working in the heart. Its safety and efficacy was unsurpassed as surgeons were able to ablate delicate cardiac structures such as the A-V node, pulmonary veins and delicate peri-nodal atrial tissue without concern for thrombosis, perforation, or other adverse events.

Cryotherapy devices deliver cryoenergy via a cryocatheter such as a balloon catheter to an area of a coronary artery where rupture of plaque has occurred (or may occur) causing a heart attack. The procedure stabilises the arterial wall by promoting healing of the area damaged by the rupture of the plaque.

A cryocatheter is designed to cool the artery tissue in the region of the balloon to a target temperature, typically between −10° C. and −20° C. Achieving the cooling power required to ensure a fast cool-down of the cryocatheter and to reach the balloon and tissue temperatures under the typical heat load in the artery is a major challenge given the design constraints on a cryocatheter: a cryocatheter must be small enough to fit into a coronary artery yet it needs to be capable of delivering sufficient cooling power.

Therefore, a problem remains in designing a method for capable of sufficiently cooling down a cryocatheter to maximise cryo-energy delivery.

### SUMMARY OF INVENTION

According to a first aspect of the invention there is provided a temperature regulation method for a balloon catheter, the method comprising supplying cryogenic fluid to a balloon of the balloon catheter in at least two phases, the at least two phases comprising a cooldown phase and a regulation phase subsequent to the cooldown phase,
wherein during the cooldown phase a supply of cryogenic fluid is delivered to the balloon to thereby cool the balloon, wherein the cooldown phase comprises:
   coupling a source of the cryogenic fluid to the balloon;
   delivering the cryogenic fluid to the balloon via a flow control valve;
   obtaining one or more temperature measurements for the balloon;
   determining, using the one or more temperature measurements, that a temperature of the balloon satisfies a first predetermined condition; and, in response to the determining, closing the flow control valve; and,
wherein during the regulation phase a flow of the cryogenic fluid to the balloon is regulated to thereby maintain a temperature of the balloon within a predetermined temperature range, wherein the regulation phase comprises repeating the following steps:
   obtaining one or more further temperature measurements for the balloon;
   determining, using the one or more further temperature measurements, whether a temperature of the balloon satisfies a second predetermined condition; and,
   if the temperature of the balloon satisfies the second predetermined condition, opening the flow control valve to increase supply of the cryogenic fluid to the balloon.

Advantageously, the method enables more effective cooling of a cryocatheter such as a balloon catheter and controlling its temperature.

Preferably, the step of delivering the cryogenic fluid in the cooldown phase comprises pulsing the flow control valve.

Advantageously, pulsing the flow control valve in the cooldown phase provides improved control over the temperature of the cryocatheter by reducing time lag between changes in delivery of the cryogenic fluid and temperature changes of the cryocatheter.

The regulation phase may comprise the step of pulsing the flow control valve with a first duty cycle and may further comprise, after the step of determining, if the temperature of the balloon does not satisfy the second predetermined condition, changing the duty cycle of the flow control valve pulsing.

The regulation phase may comprise the step of pulsing the flow control valve with a first duty cycle and may further comprise: obtaining one or more further temperature measurements for the balloon; determining, using the one or more further temperature measurements, whether a temperature of the balloon satisfies a second predetermined condition; and, if the temperature of the balloon does not satisfy the second predetermined condition, pulsing the flow control valve with a second duty cycle; and if the temperature of the balloon satisfies the second predetermined condition, opening the flow control valve to increase supply of the cryogenic fluid to the balloon.

Advantageously, pulsing the flow control valve in the regulation phase improves temperature responsiveness.

Optionally, a duty cycle of the flow control valve in the cooldown phase is higher than a duty cycle of the flow control valve in the regulation phase.

Preferably, the first predetermined condition comprises one or more of:
an internal temperature of the balloon reaching a first temperature;
a rate of change of the internal temperature of the balloon reaching a predetermined rate of change; and,
the surface temperature of the balloon reaching a second temperature, wherein the second temperature is predetermined or based on the internal temperature and/or on the rate of change of the internal temperature.

Advantageously, using these parameters as the first predetermined condition allows fine-tuning of the cooling and temperature maintenance.

Preferably, the second predetermined condition comprises one or more of:
an internal temperature of the balloon exceeding a second temperature, for instance, the internal temperature of the balloon becoming more negative than the second temperature; and,
an internal temperature of the balloon increasing or decreasing faster than a second predetermined rate of change.

Advantageously, using these parameters as the second predetermined condition allows fine-tuning of the cooling and temperature maintenance.

Preferably, the regulation phase further comprises closing the flow control valve after a pre-configured time period.

Advantageously, closing the flow control valve after a pre-configured time period improves performances of the balloon temperatures.

Preferably, the regulation phase of the method further comprises, before obtaining one or more further temperature measurements for the balloon:
closing the flow control valve;
opening the flow control valve after a predetermined time period; and
optionally, repeating the steps of closing and opening at least one more time.

Advantageously, pulsing the flow control valve at the end of the cooldown phase allows a more accurate determination of operational parameters used in the regulation phase.

Pulsing the flow control valve at the end of the cooldown phase also improves responsiveness and temperature control during the subsequent regulation phase. Preferably, the method further comprises a warm-up phase.

Advantageously, the warm-up phase enhances safety of the system in between different parts of the procedure, as well as at the end of the procedure.

Preferably, the steps of the regulation phase are repeated for predetermined duration and/or until sufficient cooling has been achieved. Preferably, the steps of the regulation phase are repeated with a frequency of between 1 Hz and 100 Hz.

Advantageously, repeating the steps of the regulation phase allows reaching the target temperature with greater control.

According to a second aspect of the invention there is provided a control apparatus for delivering cryogenic fluid to a balloon catheter according to the method described herein.

### BRIEF DESCRIPTION OF THE DRAWINGS

Examples of the present invention will now be described in detail with reference to the accompanying drawings, in which:

FIG. 1 shows a schematic drawing of a balloon catheter suitable for use with the method described herein;

FIG. 2 shows an example coolant flow circuit suitable for use with the method described herein;

FIG. 3 shows an example coolant flow circuit suitable for use with the method described herein;

FIG. 4 shows an example coolant flow circuit suitable for use with the method described herein;

FIG. 5 shows time dependence of N₂O flow (top) and tip temperature (bottom) during a test procedure;

FIG. 6 a flow diagram of the phases of operation of the control console operating according to the method described herein;

FIG. 7 shows example plots of tip temperature, balloon temperature, and cryogenic fluid flow rate during operation of the control console;

FIG. 8 shows example plots of tip temperature and cryogenic fluid flow rate during steady-state operation of the control console;

FIG. 9 shows two example scenarios of operation of the control console; and

FIG. 10 shows a schematics of a control algorithm for operating the control console.

### DETAILED DESCRIPTION

The method described herein is suitable for use with a cryotherapy system used to deliver a new treatment for stabilising ruptured or vulnerable plaque in coronary arteries. The system comprises a control console and a catheter. The console comprises a coolant flow circuit and electronic controls. The catheter is preferably a balloon catheter.

The system uses the Joule-Thomson effect, due to which expansion of a cryogenic fluid such as pressurised liquid N₂O to generate low temperatures at a distal end of the catheter, delivering cooling of the artery wall at the target location. The treatment aims to cool the artery wall to temperatures in the range of −10° C. and −20° C. for a period of time. The period of time is typically in the range of tens of seconds, preferably about 30 seconds.

### Balloon Catheter

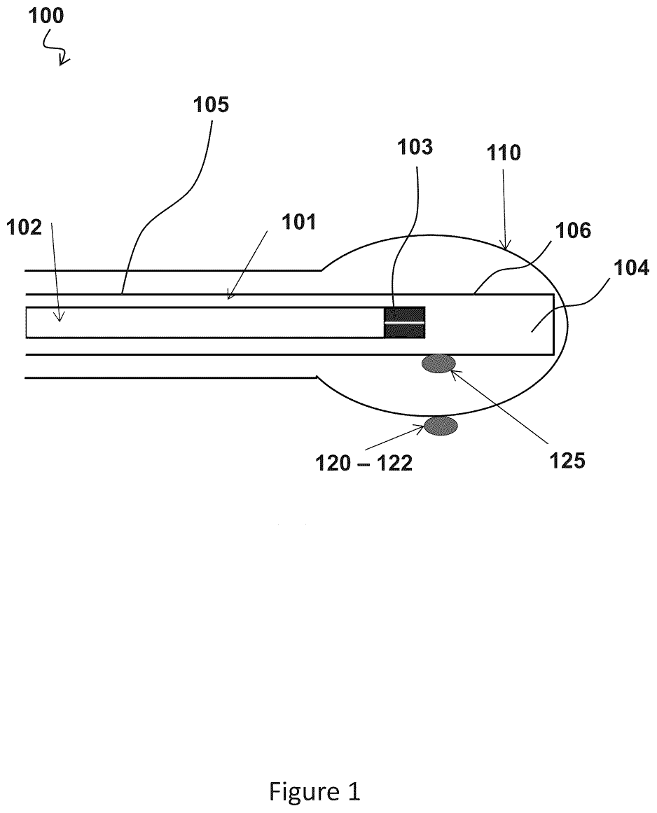

The method described here is suitable for use with a balloon catheter. An example balloon catheter 100, shown in FIG. 1, comprises a cooling element 105 and a balloon 110. The cooling element 105 comprises a tubular supply lumen 102 and a tubular return lumen 101 for the coolant. The return and supply lumen are co-axially arranged with each other, the supply lumen 102 being within the return lumen 101. An end of the supply lumen 102 is connected to and in fluid communication with a restriction tube 103. The restriction tube 103 has a narrower diameter than the supply lumen 102. The other end of the return lumen 101 to that connected to the supply lumen 102, i.e., the tip end 106, ends in an expansion chamber 104 of the cooling element 105. In the present example the expansion chamber 104 has a slightly larger diameter than the return lumen 101 and extends over the outside of the return lumen 101.

The catheter 100 also comprises at least one thermocouple 120. Preferably the catheter 100 comprises four thermocouples, 120-122, 125. Three of the thermocouples, 120-122, are mounted on the balloon 110 to monitor the balloon temperature and the temperature uniformity across the balloon 110. A tip thermocouple 125 is mounted on the outside of the return lumen 101, near the restriction tube 103, to monitor the temperature of the expansion chamber 104.

In use, a flow of pressurised coolant is input to the supply lumen 102. The coolant may be a liquid or a mixture of a liquid and a gaseous form of the coolant. The restriction tube 103 at the end of the supply lumen 102 ensures that there is little pressure drop within the supply lumen 102 and so most, or all, of the pressurised liquid coolant remains in the liquid phase in the supply lumen 102. Along the length of the restriction tube 103, the pressure drops from a maximum value at the connection to the supply lumen 102 to a lower pressure at the exit of the restriction tube 103 into the expansion chamber 104. When the liquid coolant flows into the restriction tube 103 the pressure drop caused by the restriction means that the pressure of the liquid falls below its vapour pressure at the temperature of its surroundings at that point. This causes at least some of the liquid coolant to evaporate and undergo a phase change into a gas. Liquid coolant that flows from the restriction tube 103 into the expansion chamber 104 will also expand and may evaporate within the cooling chamber 104 and/or return lumen 101. The expansion of the coolant, and the phase change of the coolant, has a cooling effect on the walls of the expansion chamber 104. The coolant then flows from the cooling chamber 104, in liquid and/or gaseous form, through the return lumen 101. The pressure within the return lumen 101, and thereby the expansion chamber 104, is preferably reduced by a vacuum pump. The vacuum pump operates on the other end of the return lumen 101 to that connected to the cooling element 105. The reduction of pressure both increases the cooling effect due to expansion and phase change of the coolant and ensures that the coolant in the expansion chamber 104 flows into the return lumen 101.

The balloon 110 is an inflatable balloon and functions as a flexible heat transfer element. One or more lumens provide supply and return flows of a fluid for inflating the balloon. These lumens are preferably separate from the lumens 101, 102 used to supply the coolant.

In use, cryogenic fluid such as N₂O from a heated pressurised gas cylinder 201 is delivered via a flow circuit 200 to the tip 106 of the catheter 100 where it expands. The fluid is then pumped out of the expansion chamber 104 through the return lumen 101 of the catheter 100 back into the console using a vacuum pump located in the console.

During the expansion at the tip 106 of the catheter 100, the fluid temperature drops significantly and cools the surrounding balloon 110 and artery.

The balloon temperature is controlled through a series of sensors and valves located on the catheter 100 and in the console. These sensors include the tip thermocouple 125 located at the tip 106 of the catheter 100, in effect on the outside of the expansion chamber 104. The sensors further include the at least one temperature sensor 120-122 on the outside of the balloon 110 and temperature sensors in the coolant flow circuit to monitor that the phase transition of the cryogenic fluid occurs at the correct temperature, for safety reasons. The sensors also include a flow sensor monitoring the return gas flow of the cryogenic fluid and pressure sensors monitoring the injection pressure and the return gas pressure. The balloon catheter may also comprise a temperature sensor.

### Coolant Flow Circuit

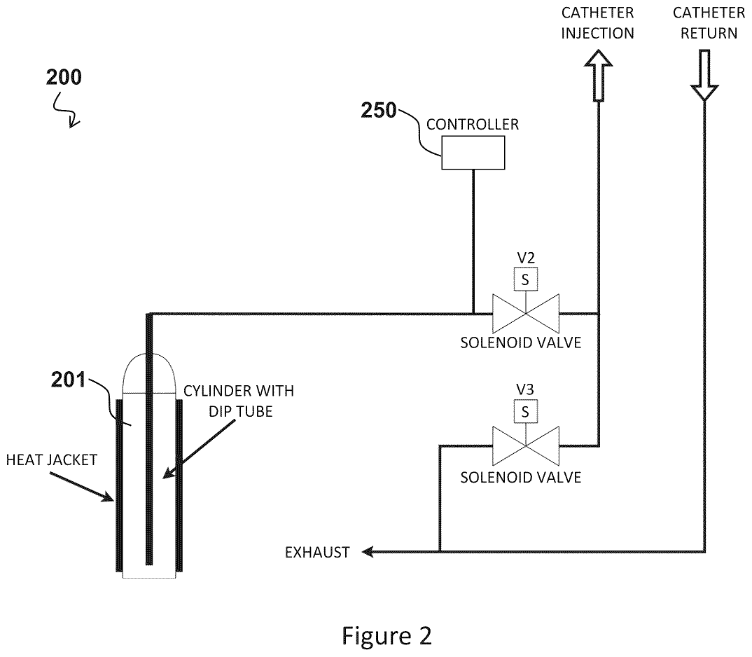

FIG. 2 shows an exemplary functional coolant flow circuit 200 suitable for use with the method described herein. The flow circuit 200 comprises a heated pressurised gas cylinder 201 for storing a cryogenic fluid such as N₂O. Heating the pressurised gas cylinder 201 raises the pressure of the cryogenic fluid inside the cylinder to the required pressure. The flow circuit 200 further comprises a flow control valve V2 and an exhaust valve V3. The flow circuit 200 is fluidly connectable to the catheter 100 to enable fluid injection to the catheter 100 and fluid return from the catheter 100.

The coolant flow circuit 200 is connected to electronic controls such as controller 250. The controller 250 controls the opening and closing of the flow control valve V2 according to the method described below. The controller 250 comprises a user interface and a display device, such as buttons foot-pedal and a tablet, to allow the user to control and monitor the procedure. The tablet may be a medical grade tablet, for regulatory purposes.

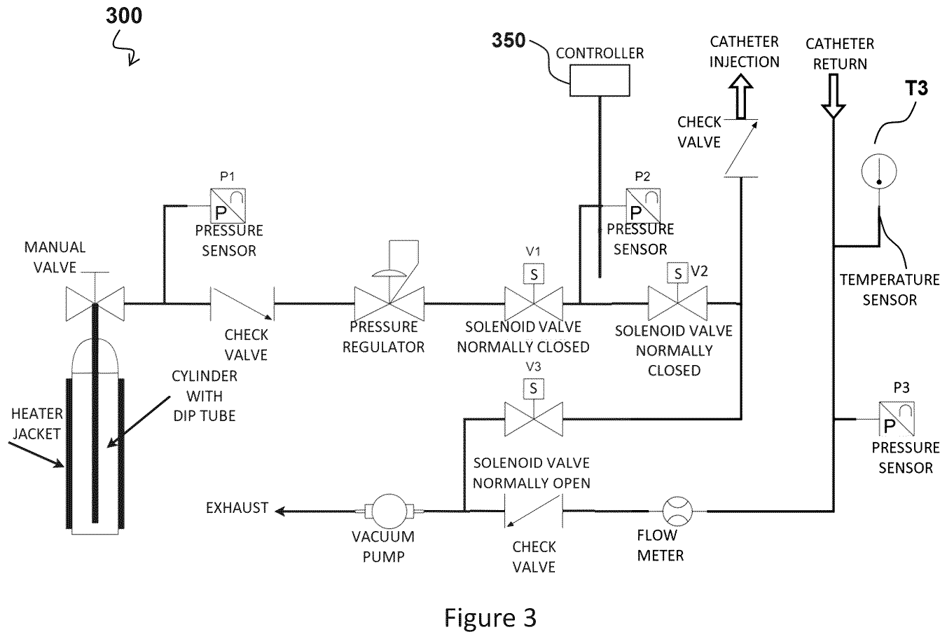

FIG. 3 shows an exemplary functional coolant flow circuit 300 suitable for use with the method described herein. The flow circuit 300 comprises the elements of the flow circuit 200. In addition, the flow circuit 300 comprises a coolant cylinder pressure sensor P1, an inlet pressure sensor P2, and a return line pressure sensor P3. The flow circuit 300 further comprises a coolant supply valve V1. The flow circuit sensor 300 further comprises a return line thermocouple T3.

The coolant flow circuit 300 is connected to electronic controls such as controller 350. The controller 350 controls the opening and closing of the flow control valve V2 according to the method described below.

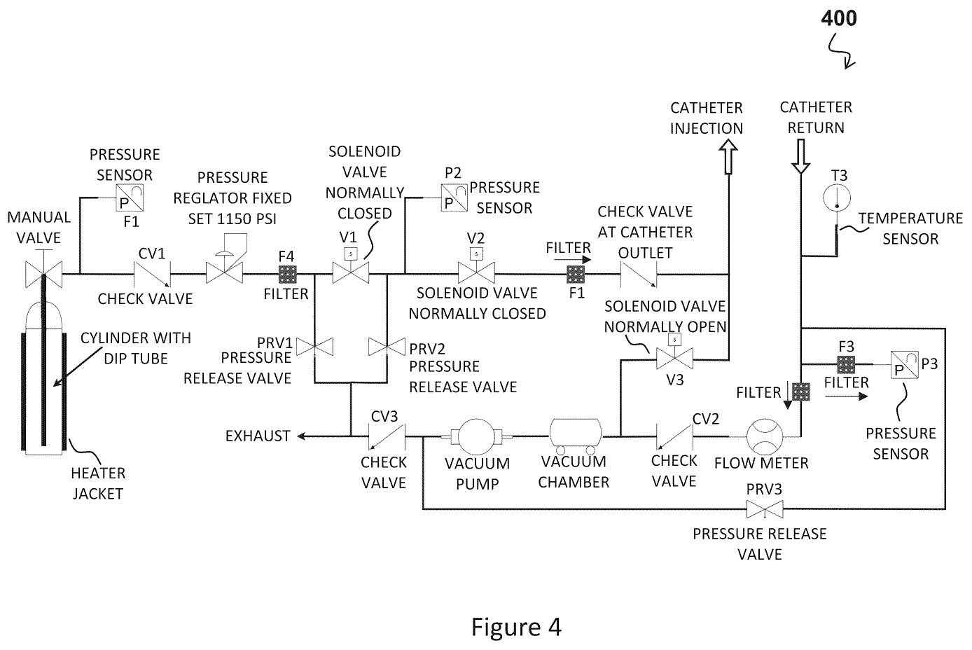

FIG. 4 shows an exemplary functional coolant flow circuit 400 suitable for use with the method described herein. The flow circuit 400 comprises the elements of the flow circuits 200 and 300.

The flow circuit 400 further comprises a first pressure relief valve PRV1, a second pressure relief valve PRV2, and a return line pressure relief valve PRV3. The first pressure relief valve PRV1 preferably has a manual override. The coolant supply valve V1 is open during normal operation.

The flow circuit 400 further comprises a coolant filter F1, a return line filter or trap F2, and a return pressure line filter or trap F3. The coolant filter F1 is connected between the flow control valve V2 and a catheter inlet to filter the cryogenic fluid before it flows into the catheter 100. The return line filter F2 is configured to filter the flow of the cryogenic fluid returning from the catheter 100. The filters improve the purity of the cryogenic fluid by removing moisture or other impurities. This lowers the risk of the fluid freezing in the circuit.

The return line filter F3 protects a pressure sensor that is embedded in the electronic controls.

The flow circuit 400 further comprises a return line flow meter FM1.

The flow circuit 400 further comprises check-valves CV1-CV3. The check-valve CV1 is connected between the pressure cylinder and the emergency stop valve V2. The check-valve CV2 is connected along the return line. The check-valves allow fluid and gas to flow in one way only. The check-valve CV1 also has a safety function in ensuring that the cryogenic fluid does not leak out of the flow circuit. The check-valve CV2 ensures that a vacuum chamber remains at negative pressure while in operation. The check-valve CV3 is connected along an exhaust line from the vacuum pump and ensures that any gas flowing out of the vacuum pump flows towards a gas exhaust and cannot return to the pump.

The flow circuit 200, 300, 400 may also comprise a coolant cylinder thermocouple T1 (not shown in the figures) connected to the gas cylinder 201 and a heater plate thermocouple T4 (not shown in the figures).

In order to control the flow of the cryogenic fluid the console uses the flow control valve V2 which opens repeatedly for short periods of time. This “bang-bang” style control approach has an advantage of being easily and readily implemented. Furthermore, the “bang-bang” style has an advantage of not introducing any further element in the system where expansion of the cryogenic fluid may occur, other than the balloon itself.

### Control Strategy

The catheter 100 is designed to cool artery tissue in the region of the balloon 110 to a target temperature between −10° C. and −20° C. Achieving the cooling power required to ensure a fast cool-down and to reach the balloon and tissue temperatures under the typical heat load in the artery is a major challenge given the design constraints on the catheter.

The control method described below plays an important role in achieving and delivering the required high level of cooling power. Furthermore, changes in the heat load can have a significant influence on the balloon temperature and cooling power. The present control method provides the ability to respond to these circumstances.

During cooling there is a temperature gradient between the expansion chamber 104 and the tissue surrounding the balloon 110. This temperature gradient will cause heat to flow from the tissue into the expansion chamber 104 where it will cause a phase change in the cryogenic fluid and heat up the return gas stream of the cryogenic fluid. The cooling power (or ability of the catheter 100 to absorb heat) is directly dependent on the temperature of the expansion chamber 104 which is measured by the tip thermocouple 125. The balloon temperatures are a direct consequence of the temperature gradient between the catheter tip 106 and the tissue and the thermal resistances across the balloon 110 and between the balloon 110 and the tissue. To achieve sufficiently low balloon temperatures (e.g., between about −20° C. and −10° C.) the tip temperature needs to be correspondingly low (e.g., between about −60° C. and −40° C.).

Achieving low tip and balloon temperatures is the result of a trade off. On one hand the maximum cooling power the catheter 100 could deliver is proportional to the mass flow rate of the cryogenic fluid through the catheter 100; therefore a sufficiently high flow rate is required to deliver a high cooling power. On the other hand the boiling point of the cryogenic fluid (such as N₂O) inside the expansion chamber 104 is a function of the pressure in this chamber. This pressure is determined by the pressure drop across the return flow path of the cryogenic fluid in gas form. For a given catheter and a given console this pressure drop depends on the flow rate of the cryogenic fluid. In order to achieve a low pressure drop and low boiling temperature, a low flow rate is desired.

Given these two counter-acting factors, there is an optimal cryogenic fluid flow rate for a given heat load. In order to achieve a tip temperature within a range that leads to the balloon and tissue temperatures in the required ranges, the cryogenic fluid flow rate needs to be such that the control algorithm is able to balance the cooling power from the cooling liquid expansion with the heat load and maintain this temperature gradient across the balloon. Providing a lower flow rate will result in the catheter not being able to provide sufficient cooling power and the tip temperature will therefore rise. Providing a higher flow rate than required will cause the N₂O to build up in the return flow path causing a pressure increase in the return flow path, which in turn will lead to smaller expansion in the balloon.

In other words, a flow rate that is higher than required will cause the pressure drop across the return flow path to be lower and will therefore result in a higher boiling point of the cooling liquid and higher tip temperature. In this case the return gas will carry away the excess cooling power and will therefore be cooler.

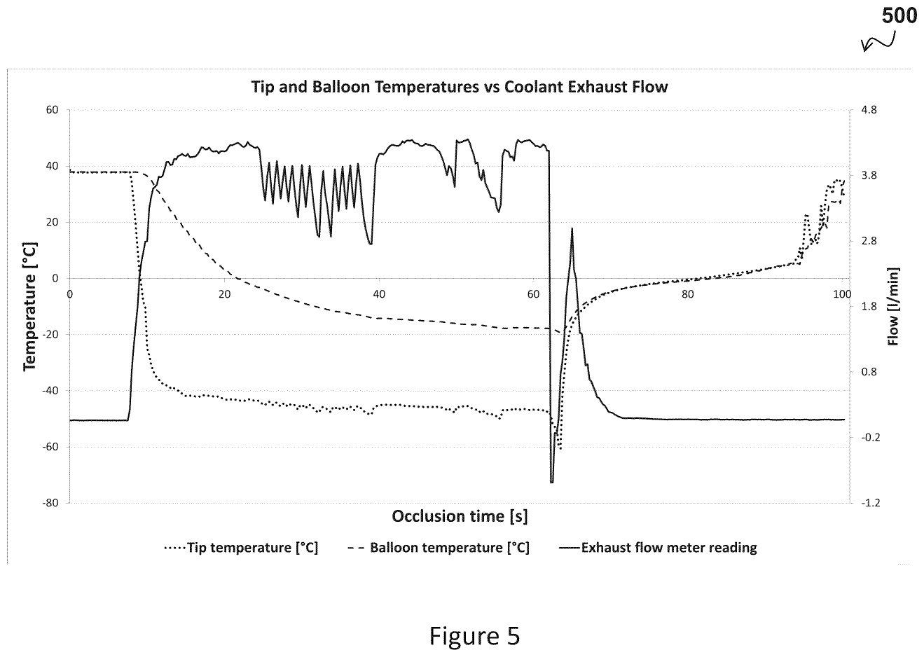

FIG. 5 illustrates this balance. The plot 500, shows the time dependence of N₂O flow and the temperatures of the tip and the balloon, respectively, during a test procedure. After the initial cool-down, a high flow rate of about 4 l/min is maintained. This results in a tip temperature of about −20° C. This flow rate is too high for the heat load on the catheter and reducing the flow rate to about 2 l/min results in a drop of the tip temperature by about 10° C., as shown in plot 500 of FIG. 5. However, when the flow rate is reduced further below about 2 l/min, the tip temperature increases significantly as the N₂O expansion is no longer able to provide sufficient cooling to balance the heat load on the catheter at this low tip temperature.

Achieving and managing the correct balance is required for each catheter and procedure. In addition, during a procedure, the heat load on the catheter may change and hence this balance needs to be continuously monitored and maintained.

The control approach described below does not regulate the balloon temperature, but instead aims to achieve a very low tip temperature. Achieving a very low tip temperature will ensure that the balloon temperature is as low as possible. In addition, the control system will also ensure that balloon temperatures below −20° C. are detected and acted upon in order to ensure patient safety.

### Phases of Operation

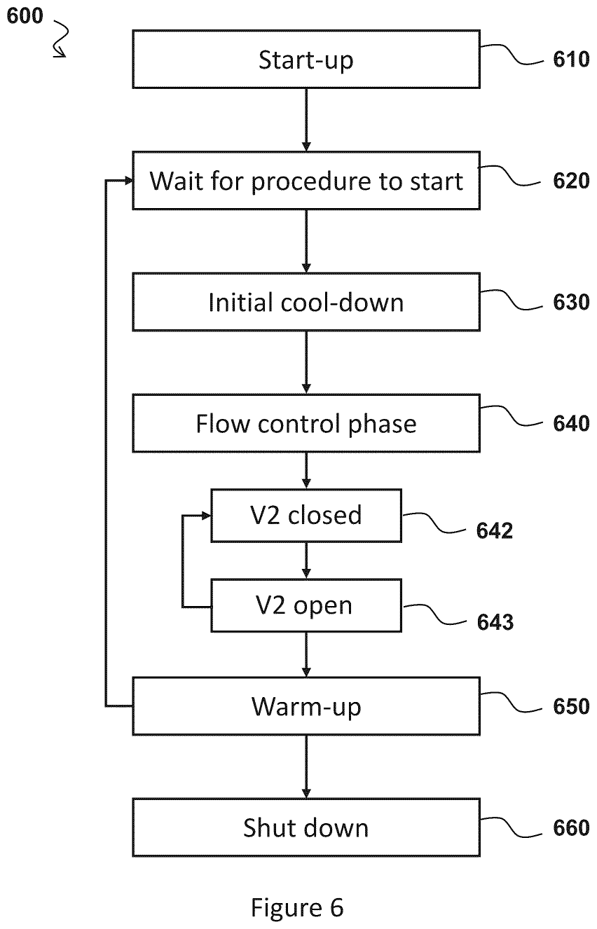

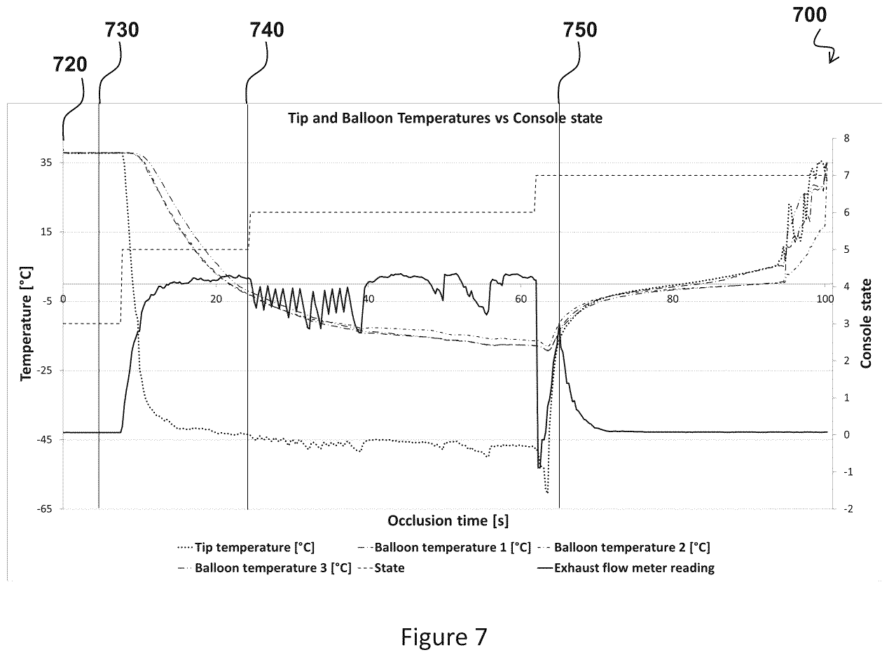

FIG. 6 shows a flow diagram 600 of the six phases of operation of the console operating according to the method described here. FIG. 7 shows plots 700 of the tip temperature, balloon temperature, and the flow rate during the middle four phases of the operation.

### 1. Start-Up Phase 610

In the start-up phase 610 the console is powered up and the catheter 100 is connected to the flow circuit. Start-up tests of the components of the system are also initiated, to ensure that all components are in good working order and all conditions are set up for safe operation of the device.

### 2. Wait for the Start of the Procedure 620

This phase encompasses the period between the completion of the start-up phase and the start of the treatment. Certain system parameters will need to be maintained and monitored to ensure the console is working as intended and ready for the treatment of the patient.

This phase assumes that the console has already achieved the following start condition prior to entering this phase. All start-up checks have been successfully completed. The catheter 100 is connected to the console. The cylinder has been heated up to operating temperature and has achieved operating injection pressure within a desired pressure interval, as measured by pressure sensor P1. The cylinder heating is maintaining the cylinder temperature at the start of this phase. Further, at the start of this phase, the flow control valve V2 is closed, while the coolant supply valve V1 (if present) and the exhaust valve V3 are open. The vacuum pump is on and the return pressure is in an expected pressure range. The flow rate of the cryogenic liquid is substantially zero.

The start of this phase is denoted 720 in the plots of FIG. 7. The tip temperature and the balloon temperature remain steady, while the flow rate is substantially zero. The cardiologist performing the treatment will initiate the procedure by pressing the foot pedal.

### 3. Initial Cool-Down Phase 630

During this phase the balloon 110 is cooled to a temperature in the treatment range. In the absence of any fault condition there should be no delay in moving from the wait for the procedure to start phase 620 to the initial cool-down phase 630 once the foot pedal is pressed to signal the start of the coolant flow.

At the start of this phase the flow control valve V2 is closed, while the coolant supply valve V1 and the exhaust valve V3 are open. Then the flow control valve V2 is opened to start the cryogenic fluid delivery and begin cooling down the catheter tip 106. The cool-down of the catheter tip 106 is monitored. Various parameters may be measured during this phase, for instance the tip temperature, the balloon temperature, the cryogenic liquid flow rate, cylinder pressure in the cryogenic fluid source, and/or injection pressure. Further parameters, such as the state of the foot pedal, the state of a stop button of the console, fault conditions, and whether the balloon 110 is occluded (i.e., not inflated), may also be monitored.

Typically, this phase has two stages. In the first cool-down stage 631 the catheter tip 106 is cooled down, whereas in the second cool-down stage 632 the balloon 110 is also cooled down. The target control values for the following flow control phase 640 are defined by the parameters measured in this phase.

In the cool-down phase 630 the cryogenic fluid may be delivered continuously or by pulsing the flow control valve V2. Continuous, or “full throttle”, delivery may provide faster cooling. However, “full throttle” delivery may lead to saturation of the catheter tip with the cryogenic fluid. If the delivery of the cooling fluid is stopped while the catheter tip is saturated, the effects on the temperature of the catheter tip are delayed. This delay may be on the order of seconds, for instance up to 15 seconds. Delivering the cryogenic fluid by pulsing the flow control valve V2 avoids this saturation and thus ensures the temperature of the catheter tip is more responsive. This effect is particularly pronounced when the temperature has decreased quickly.

The start of this phase is denoted 730 in the plots of FIG. 7. As shown in the plots, during this phase the tip and balloon temperatures drop and the flow rate initially increases and then remains substantially steady for the remainder of this phase.

### 4. Flow Control Phase 640

In the flow control phase 640 the tip temperature is maintained at a low level and the balloon temperature is maintained at a temperature in the treatment range. The tip temperature will be actively controlled using the control algorithm. Checks may be included for the balloon temperature, flow rate, return temperature, inlet and outlet pressures and other parameters in order to ensure safe operation and detection of device failures. In addition, checks on occlusion and procedure times may be performed. The start of this phase is denoted 740 in the plots of FIG. 7.

In the absence of any fault condition there should be no delay in moving from the initial cool-down phase 630 to the flow control phase 640 once the conditions for the start of the flow control phase 640 have been met.

At the start of this phase the coolant supply valve V1 and the flow control valve V2 are open, while the exhaust valve V3 is closed. The flow control valve V2 is then closed. During this phase the flow control valve V2 opening and closing is controlled to maintain and reduce the tip temperature and to achieve a sufficient cooling rate.

During this phase the average tip temperature is calculated, preferably using an exponential filter algorithm:

Tav denotes an average tip temperature, Tₜₙ the tip temperature at time tₙ, and fo is a weighting factor. The updated value of Tav outputted at the last step of the algorithm is a weighted sum of two contributions, i.e., the input Tav and the tip temperature tₙ at a given time. The parameter f0 distributes the weights between these two contributions. The larger the value of f0 is, the larger the contribution of the input Tav will be in the output Tav value relative to the contribution of the tip temperature tₙ.

If certain conditions, described in more detail in the following section, are met, the flow control valve V2 is pulsed during this phase. In some embodiments the flow control valve V2 is controlled with a pulse width modulation (PWM) approach in the cool-down phase 630 and/or in the regulation phase 640. Pulsing the flow control valve V2 in the cool-down phase 630 and/or in the regulation phase 640 improves cooling efficiency by focussing cooling on the balloon. In contrast, a proportional valve would provide cooling power to parts of the system other than the balloon instead of concentrating the cooling. In other words, this would likely cause expansion of the cryogenic fluid at the needle of the proportional valve or at any other restriction in the system, therefore providing cryoenergy to parts of the system other than the cryocatheter. Thus, pulsing the flow control valve V2 improves cooling efficiency compared to using a proportional valve.

The duty cycle of the pulses used in the cool-down phase 630 and in the regulation phase 640 may be different. For instance, in the cool-down phase 630 the duty cycle may be higher than in the regulation phase 640. A duty cycle of 100% corresponds to a continuous delivery of the cryogenic fluid. In the cool-down phase 630 the duty cycle may be at least 0.1%. In the cool-down phase 630 the duty cycle may be up to 100%.

In the regulation phase 640 a pre-defined duty cycle is chosen depending on a pre-defined temperature threshold, in contrast to a proportional integral derivative (PID) control algorithm. Different duty cycles can be pre-defined for different temperature ranges or temperature thresholds. For example, if the temperature is in the expected range, i.e., between about −10° C. and −20° C., and the temperature is further decreasing, the system may reduce the duty cycle. If the temperature is in the expected range, i.e., between about −10° C. and −20° C., and the rate of change of the temperature is below a threshold, the system may keep the current duty cycle. In another example, if the rate of change of the temperature is positive, the system may increase the duty cycle. Thus, more than one duty cycle (which can include a first duty cycle and a second duty cycle) may be used during the regulation phase, depending on the specific conditions. In contrast to a PID controller, there is not a single target temperature, but rather a range of temperatures, which enables the use of more complex control logic in the decision making tree.

If during the cool-down phase 630 the rate of change of the tip or balloon temperature is lower than a threshold rate, the system may transition to the regulation phase 640 where the duty cycle is controlled as described above.

During this phase the state of the foot pedal may be monitored, the state of the stop button of the console may be monitored, and fault conditions are preferably monitored.

Optionally, at the beginning of this phase the flow control valve V2 may be pulsed once or several times and/or for a predetermined length of time. If after the pulsing the flow control valve V2 the tip temperature moves closer to the target temperature the flow control valve V2 is kept in pulsing mode and the method proceeds with the flow control phase 640. If after the pulsing the tip temperature remains the same or moves further away from the target temperature, the flow control valve V2 is kept steadily open. The pulsing may be used to determine operating parameters for the flow control phase 640.

### 5. Warm-Up Phase 650

The start of this phase is denoted 750 in the plots of FIG. 7. Typically, the cardiologist signals the end of the procedure by a second pressing of the foot pedal. At the start of this phase the coolant supply valve V1 is open, while the flow control valve V2 and the exhaust valve V3 is closed. At this time the coolant flow is stopped and the catheter 100 is allowed to warm up. The exhaust valve V3 may be opened to pump the cryogenic fluid out of the catheter 100 and the catheter connections in order to speed up the warming of the catheter. During the warm-up phase 650 various system parameters are monitored to ensure safe operation of the system. On completion of the procedure, the console may return to the wait for the start of the procedure phase 620, or it may proceed to a shut down phase 660.

One of the requirements of a medical intervention such as cryotherapy is to keep the intervention as short as possible, to avoid stopping the blood flow of the patient for a long time, as that could have adverse consequences for the patient. A treatment may last for instance for about 30 seconds. When the treatment is finished, the blood vessel should be brought back to the original temperature as soon as possible. In use, once the catheter 100 is filled with the cryogenic fluid, the fluid expands at a relatively low rate in the tip 106 and is vented out through the return line. It may also be extracted by the vacuum pump. The exhaust valve V3 allows to remove the excess cryogenic fluid (trapped in the part of the piping between the control valve V2 and the catheter tip 106) directly from the injection line. The residual coolant is therefore not forced to flow through the catheter 100 in order to be vented out but is evacuated much faster through the exhaust valve V3. This leads to significant shortening of the warm-up phase 650.

In other words, if the exhaust valve V3 did not open after the treatment is finished, there would be a significant amount of the cryogenic fluid that would have to flow into and out of the catheter 100, still producing some degree of cooling in the vessel and therefore significantly delaying the time when the surgeon can remove the catheter 100 from the patient so that regular blood flow can be restored. The addition of the exhaust valve V3 has been specifically designed for the method described here, because without the valve V3 the timing was too slow and not compatible with the requirements of this type of surgery.

### 6. Shut Down Phase 660

Once all procedures have been completed, the system is shut down. Preferably, the system should be shut down in a safe manner and made ready for storage until the next start-up.

Optionally, additional phases may be required to deal with failure handling and system maintenance.

### Flow Rate Control in the Flow Control Phase 640

Controlling the flow rate is important for achieving and maintaining a low tip temperature. Known approaches, such as using an electronic pressure regulator or needle valve to control the cryogenic fluid flow rate have the disadvantage of being too expensive compared to the method described here. Moreover, with these known approaches the pressure drop across the flow control device used, e.g. the electronic pressure regulator or the needle valve, would need to be significantly larger than the pressure drop across the restriction tube 103 at the end of the supply lumen 102. This significantly larger pressure drop would in turn reduce the cooling power available. Furthermore, electronic pressure regulators and needle valves typically have slow response times due to the large gas volumes in the system.

In contrast, the method described here overcomes these issues. The approach uses a valve, such as the flow control valve V2, to implement a bang-bang control system. The valve is preferably a solenoid valve. The valve may also be a manual, pneumatic, or hydraulic valve. This approach is characterised by a low flow resistance and pressure drop across the flow control valve V2 in the on-state.

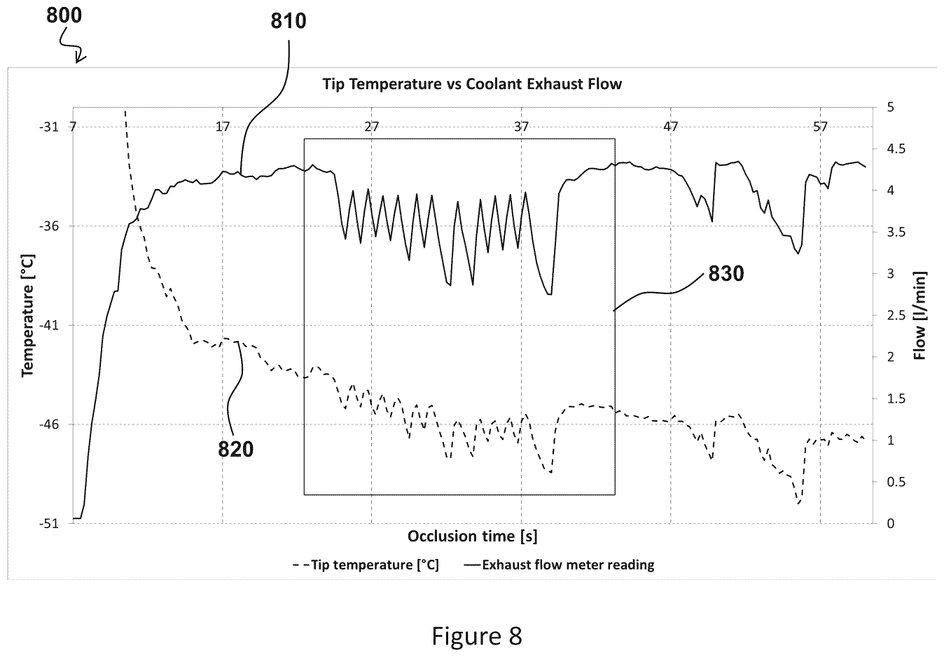

FIG. 8 shows the changes in flow rate 810 and tip temperature 820 over time. Rectangle 830 marks the steady-state operation in the flow control phase 640.

In steady-state operation a typical control cycle comprises the following steps. At step 641 the flow control valve V2 is opened for a short period of time t_open1, typically on the order of hundreds of milliseconds, for instance on the order of or less than 100 ms, 500 ms, and/or less than 10% of the duration of the flow rate control phase 640. During this on-state the volume of cryogenic fluid in pipework leading to the catheter 100 and the supply lumen 102 is filled with the cryogenic fluid and pressurised up to the cylinder pressure.

At step 642 the flow control valve V2 is closed. During this off-state, the cryogenic fluid stored in the pipework between the flow control valve V2 and the catheter tip 106 flows through the catheter tip 106, reducing the cryogenic fluid pressure in the pipework. As this pressure decays, the cryogenic fluid flow rate reduces and the tip temperature reduces. As the pressure drops further, the cryogenic fluid flow rate is no longer sufficient to provide the required cooling power and this typically leads to an increase in the tip temperature.

At step 643 the flow control valve V2 is opened again for the short period of time t_open2, when the flow rate drops below a trigger value or when certain conditions relating to the flow rate or tip temperature are fulfilled in order to maintain a low tip temperature. The whole cycle is repeated, with step 643 being analogous to step 641.

The lowest tip temperature that can be achieved using this approach depends on the detection of the correct time to open the control valve, the valve opening time t_open1, t_open2, and the heat load on the system. The tip temperature then rises as the cooling liquid flow rate decreases below the minimum level required to maintain the tip temperature, as shown in FIG. 5. The temperature overshoot observed depends on the timing of the flow control valve V2 opening and the heat load, with larger overshoots typically observed for lower heat loads.

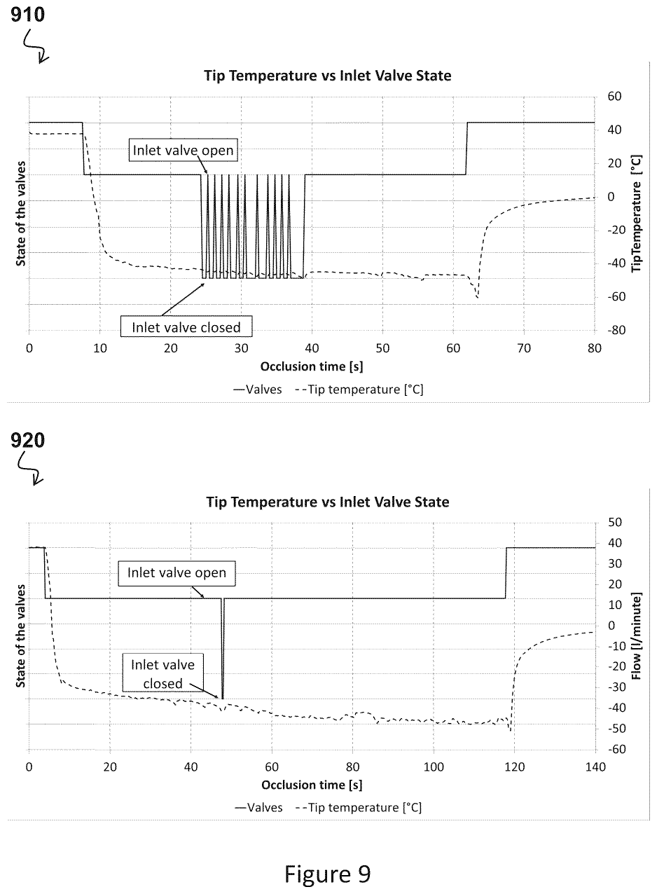

FIG. 9 illustrates two different scenarios of operation of the console. In plot 910 a lower tip temperature and therefore also lower balloon temperatures can be achieved when the cryogenic fluid flow is reduced after the cool-down phase and when the flow rate control (steps 641-643) is implemented. In these cases, pulsing the flow control valve V2 to reduce the flow is beneficial and leads to a lower average tip temperature.

However, there are also cases, for example when the catheter is subjected to a very high heat load, where the closing of the flow control valve V2 and the subsequent reduction in the flow rate will lead to an increase in the average tip temperature. An example case is shown in plot 920 of FIG. 9. In these cases closing the flow control valve V2 can still lead to a momentary reduction in the tip temperature which is then followed by a rapid increase as the cryogenic fluid flow rate is no longer sufficient to maintain the required cooling power. In these cases it is more beneficial to keep the flow control valve V2 open and to operate the catheter 100 at the maximum flow rate possible for given injection pressure, so as to achieve a lower average tip temperature.

### Control Algorithm

In practice the operation of the console may be controlled by a control algorithm. The algorithm deals mainly with the wait for the start of the procedure phase 620, the initial cool-down phase 630, the flow control phase 640, and the warm-up phase 650.

In the flow control phase 640 the control algorithm evaluates whether pulsing the control valve pulsing the flow control valve V2 will lead to a lower average tip temperature or not and based on the evaluation implements the most beneficial strategy to achieve an average tip temperature in the required range.

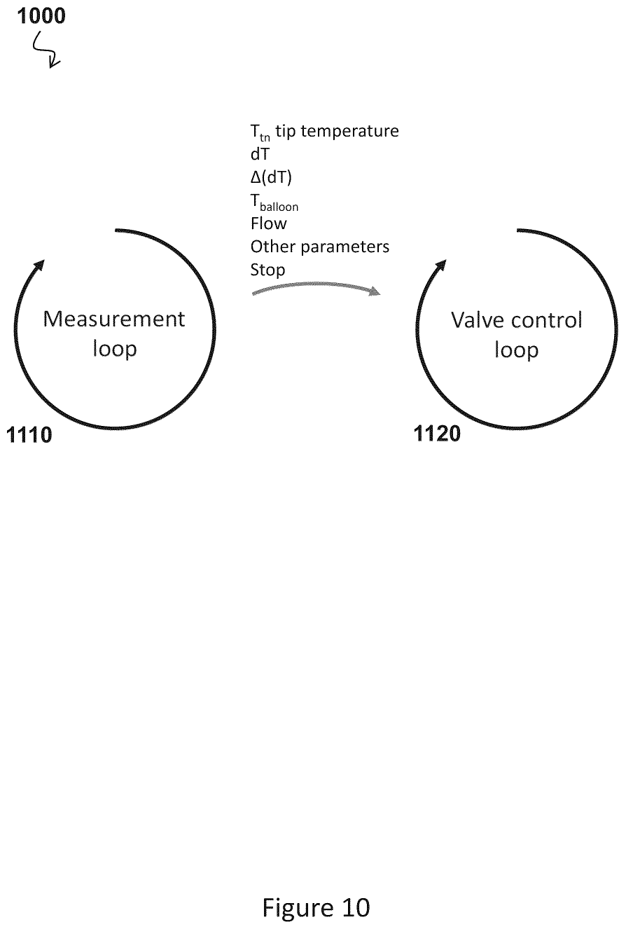

Schematics 1000 in FIG. 10 shows the main elements involved in the control algorithm. The algorithm comprises a measurement loop 1010, which measures a range of parameters over a measurement interval and calculates a number of derived parameters. The loop passes these on to a valve control loop 1020. In order to measure a particular parameter value, measurements of the corresponding sensor output may be taken at a high sample rate over the measurement interval and then averaged over the measurement interval to provide the parameter value. Preferably the sample rate is up to 100 Hz. Other sample rates may be used, for instance about 10 Hz, 50 Hz, 200 Hz, 1 kHz, 10 KHz, or 50 kHz.

As an example, the measurement loop may take one set of measurements every 200 ms or every 250 ms.

The valve control loop 1020 makes decisions on the opening and closing of the flow control valve V2 and, where appropriate, other valves based on the measurements made by the measurement loop. The decisions made by the valve control loop also depend on the phase of operation.

The two loops 1010, 1020 operate in a synchronous manner. The measurement loop 1010 measures a set of parameter values and passes these parameter values on to the valve control loop 1020 which makes a decision on the basis of these parameter values. The data hand-over point may be used to synchronise the loops with each other. This process is then repeated.

Example main parameters measured by the measurement loop 1010 to support the valve control loop decisions are tip temperature, rate of change of the tip temperature, at least one balloon temperature as measured by the at least one balloon thermocouple 120-122, return pressure, pressure within the balloon, and/or flow rate of the cryogenic fluid.

1. A temperature regulation method for a balloon catheter, the method comprising supplying cryogenic fluid to a balloon of the balloon catheter in at least two phases, the at least two phases comprising a cooldown phase and a regulation phase subsequent to the cooldown phase,
wherein the cooldown phase comprises:
coupling a source of the cryogenic fluid to the balloon;
delivering the cryogenic fluid to the balloon via a flow control valve to cool the balloon;
obtaining one or more temperature measurements for the balloon;
determining, using the one or more temperature measurements, that a temperature of the balloon satisfies a first predetermined condition; and,
in response to the determining, closing the flow control valve; and,
wherein the regulation phase comprises repeating the following steps:
obtaining one or more further temperature measurements for the balloon;
determining, using the one or more further temperature measurements, whether a temperature of the balloon satisfies a second predetermined condition; and,
if the temperature of the balloon satisfies the second predetermined condition, opening the flow control valve to increase supply of the cryogenic fluid to the balloon.

2. The temperature regulation method of claim 1, wherein the step of delivering the cryogenic fluid in the cooldown phase comprises pulsing the flow control valve.

3. The temperature regulation method of claim 1, wherein the regulation phase comprises the step of pulsing the flow control valve with a first duty cycle and further comprises:
obtaining one or more further temperature measurements for the balloon;
determining, using the one or more further temperature measurements, whether a temperature of the balloon satisfies a second predetermined condition; and,
if the temperature of the balloon does not satisfy the second predetermined condition, pulsing the flow control valve with a second duty cycle; and
if the temperature of the balloon satisfies the second predetermined condition, opening the flow control valve to increase supply of the cryogenic fluid to the balloon.

4. The temperature regulation method of claim 3, wherein a duty cycle of the flow control valve in the cooldown phase is higher than a duty cycle of the flow control valve in the regulation phase.

5. The temperature regulation method of claim 1, wherein the first predetermined condition comprises one or more of:
an internal temperature of the balloon reaching a first temperature;
a rate of change of the internal temperature of the balloon reaching a predetermined rate of change; and,
the surface temperature of the balloon reaching a second temperature, wherein the second temperature is predetermined or based on the internal temperature and/or on the rate of change of the internal temperature.

6. The temperature regulation method of claim 1, wherein the second predetermined condition comprises one or more of:
an internal temperature of the balloon exceeding a second temperature; and,
an internal temperature of the balloon increasing or decreasing faster than a second predetermined rate of change.

7. The temperature regulation method of claim 1, wherein the regulation phase further comprises closing the flow control valve after a pre-configured time period.

8. The temperature regulation method of claim 1, wherein the regulation phase further comprises, before obtaining one or more further temperature measurements for the balloon:
closing the flow control valve;
opening the flow control valve after a predetermined time period; and optionally, repeating the steps of closing and opening at least one more time.

9. The temperature regulation method of claim 1, further comprising a warm-up phase following the regulation phase.

10. The temperature regulation method of claim 9, wherein during the warm-up phase an exhaust valve is open to allow venting out residual cryogenic fluid from the control apparatus.

11. The temperature regulation method of claim 1, wherein the steps of the regulation phase are repeated for predetermined duration and/or until sufficient cooling has been achieved.

12. The temperature regulation method of claim 1, wherein the steps of the regulation phase are repeated with a frequency of between 1 Hz and 100 Hz.

13. A control apparatus for delivering cryogenic fluid to a balloon catheter, the control apparatus comprising:
a flow control valve fluidly connectable to the balloon catheter to deliver the cryogenic fluid; and
a processor storing computer executable instructions for performing the steps of the method of claim 1 by controlling flow of the cryogenic fluid to the balloon catheter via the flow control valve,
wherein the control apparatus is fluidly connectable to a pressurised cylinder for supply of cryogenic fluid.

14. The control apparatus of claim 13, further comprising an exhaust valve, fluidly connectable to the balloon catheter, wherein the exhaust valve is configured to vent out residual cryogenic fluid from the control apparatus.

15. The control apparatus of claim 13, further comprising a coolant supply valve, configured to fluidly connect the flow control valve and the pressurised cylinder.

16. The control apparatus of claim 13, further comprising:
a coolant cylinder pressure sensor, configured to measure operating injection pressure;
an inlet pressure sensor, configured to measure pressure of the cryogenic fluid before it enters the flow control valve; and a return line pressure sensor, configured to measure pressure of the cryogenic fluid returned from the balloon catheter.

17. The control apparatus of claim 13, further comprising at least one of a first pressure relief valve, a second pressure relief valve, and a return line pressure relief valve, wherein the first pressure relief valve preferably has a manual override.

18. The control apparatus of claim 13, further comprising at least one of a coolant filter, a return line filter or trap, and a return pressure line filter or trap.

19. (canceled)
